(19) United States
(12) Patent Application Publication
(10) Pub. No.: US 2015/0203466 A1
(43) Pub. Date: Jul. 23, 2015

(54) Trans-2-nonadecyl-4-hydroxymethyl-1,3-dioxolane and method for producing thereof

(71) Applicants: Min-Hsiung PAN, Kaohsiung (TW); Greenyn Biotechnology Co., Ltd, Taichung (TW)

(72) Inventors: Min-Hsiung PAN, Kaohsiung (TW); Chi-I CHANG, Pingtung County (TW); Chia-Feng WU, Taichung (TW); Pang-Kuei HSU, Taichung (TW)

(21) Appl. No.: 14/456,066

(22) Filed: Aug. 11, 2014

(30) Foreign Application Priority Data
Jan. 23, 2014 (TW) 103102539

(51) Int. Cl. C07D 317/20

(52) U.S. Cl. CPC C07D 317/20

(57) ABSTRACT

The present invention discloses a method for producing trans-2-nonadecyl-4-hydroxymethyl-1,3-dioxolane comprising mixing alkanes and pyridinium chlorochromat for an oxidation reaction; obtaining a product by the oxidation reaction for processing an aldolization reaction and then purifying for obtaining a trans-2-nonadecyl-4-hydroxymethyl-1,3-dioxolane compound. According to the method of the present invention, it is able to promote the productivity and reduce the cost.

### FIELD OF THE INVENTION

The present invention relates to a compound and method for producing thereof, especially relates to a trans-2-nonadecyl-4-hydroxymethyl-1,3-dioxolane and method for producing thereof.

### DESCRIPTION OF THE RELATED ART

Liver diseases have apparently become an important concern to modern people. More specifically, the factors like alcohol, medicine, drug or virus can cause the chronic inflammation of liver parenchyma continuously or repeatedly happened. It results in lasting damages, even necrosis, about liver cells and engages the individual in high risks of liver diseases like fatty liver or liver cirrhosis, even has an extremely high risk to become liver cancer. Liver fibrosis is a pathological symptom of chronic liver damage. The main cause lies on proliferation and decomposition of liver fibrillar connective tissues which were under an unbalance status and over-accumulated liver extracellular matrixes when liver cells suffered last and long damages. Liver fibrosis is also an essential process for that the chronic liver diseases turn to the liver cirrhosis or the liver cancer. Liver fibrosis is not an irreversible process, so there are few effective drugs, such as colchicine, adrenal cortex hormones and interferon, provided to treat it. However, said drugs can't performance free from side effects and can merely block partial symptoms of liver fibrosis, but not to inhibit all of it. Besides of western medicines, there are many Chinese herbs to treat liver fibrosis in traditional Chinese medicine, for examples, danshen root, radix notoginseng, turtle shell, starwort root, etc. Chinese herbs can decrease side effect to the individual, but it have to take times to perform drug effect, lead to more costs, and the effectiveness of Chinese herbs will be disturbed by jumbled ingredient from unknown sources.

Besides, some kinds of food have immune enhance function to treat liver diseases, generally like clams. However, it needs to eat large amount of clams to perform the effectiveness. So, there are clinical difficulties to treat diseases. In order to avoid said defects, Taiwan patent no. 1370747 revealed a method to purify the extraction of clams from alcohol in column to extract a compound which is active to against liver fibrosis. But the method described in the prior art needs to gather the active compound through complicated processes. In other words, the method revealed in the prior art includes following problems. First, it needs large amount of clams as materials and leads to extremely high costs of production. Second, it needs at least two steps to purify the active compound, so the productivity is too low.

Therefore, it still lacks an effective compound which has specific effectiveness for liver fibrosis and less side effect in revealed prior art. So far, the most important subject is to develop a compound which is low production costs, less side effect and effective to inhibit liver fibrosis for clinical treatment of liver diseases.

### SUMMARY OF THE INVENTION

The major purpose of the present invention is to provide a trans-2-nonadecyl-4-hydroxymethyl-1,3-dioxolane or pharmaceutically acceptable salt thereof, wherein the trans-2-nonadecyl-4-hydroxymethyl-1,3-dioxolane compound of formula (I);

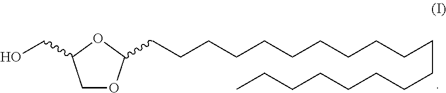 [SMILES: CCCCCCCCCCCCCCCCCCCC1OCC(CO)O1]

Another purpose of the present invention is to provide a method to produce the trans-2-nonadecyl-4-hydroxymethyl-1,3-dioxolane or pharmaceutically acceptable salt thereof, comprising producing a mixture containing the compound of formula (I) by acetalization reaction of the compounds of formula (II):

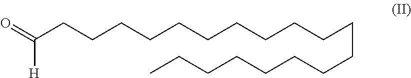 [SMILES: CCCCCCCCCCCCCCCCCCCC=O]

and formula (III):

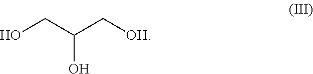

In an embodiment of this present invention, the compound of formula (II) is produced by the oxidation reaction of the compound of the formula (IV):

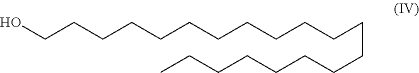 [SMILES: CCCCCCCCCCCCCCCCCCCCO]

and the pyridinium chlorochromate in organic solvent.

In an embodiment of this present invention, an acidic catalyst is used during the acetalization reaction.

In an embodiment of this present invention, the mixture containing the compound of formula (I) can be purified by separation process, wherein the isolation process includes, but not limited to, chromatography, salt out or other method which is recognized by a person of ordinary skill in the art.

### BRIEF DESCRIPTION OF THE DRAWINGS

FIG. 1 is the ¹H-NMR spectrogram of the active material from example 2 in this present invention.

FIG. 2 is the ¹³C-NMR spectrogram of the active material from example 2 in this present invention.

FIG. 3 is the mass spectrogram of the active material from example 2 in this present invention.

### DETAILED DESCRIPTION OF THE INVENTION

The present invention is further described with the following examples and figures.

### Example 1

### Production of the Compound of Formula (I)

Firstly, equal amount of the compound of formula (IV):

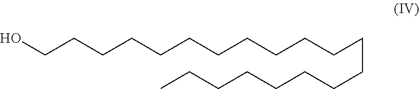 [SMILES: CCCCCCCCCCCCCCCCCCCCO]

and pyridinium chlorochromate (hereafter referred to as PCC) were dissolved in dichloromethane, and then stirred and mixed it all together, wherein, at this present example, the amount of the compound of formula (IV) or PCC was 1 gram, and the amount of dichloromethane was 20 ml. The dichloromethane solution containing the compound of formula (IV) and PCC was suction filtered through Celite to obtain a filtrate, and then the filtrate was condensed under a reduced pressure to have the compound of formula (II):

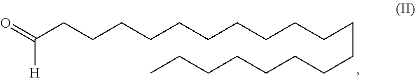 [SMILES: CCCCCCCCCCCCCCCCCCCC=O]

which was a white solid. The aforesaid reaction is shown as the following chemical equation:

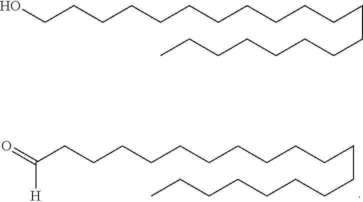 [SMILES: CCCCCCCCCCCCCCCCCCCCO ; CCCCCCCCCCCCCCCCCCCC=O]

50 ml of toluene was as solvent to dissolve the compound of formula (II), and then added 700 mg of the compound of the formula (III):

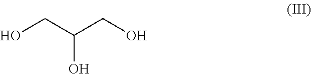

and 300 mg of p-toluenesulfonic acid (TsOH). The toluene solution containing the compound of formula (II), the compound of the formula (III) and p-toluenesulfonic acid (TsOH) was heated to reflux for about 3 hours. After that, the toluene solution was removed the toluene therein by condensing under a reduced pressure, then added aqueous solution of sodium carbonate (Na₂CO₃) containing 1% by weight of sodium carbonate for 20 ml and then extracted 3 times by ethyl acetate. The extracts were collected and removed ethyl acetate by condensing under a reduced pressure to have a mixture containing the compound of formula (I)

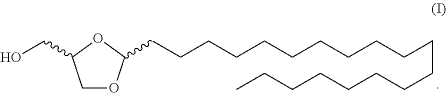 [SMILES: CCCCCCCCCCCCCCCCCCCC1OCC(CO)O1]

The aforesaid reaction is shown as the following chemical equation:

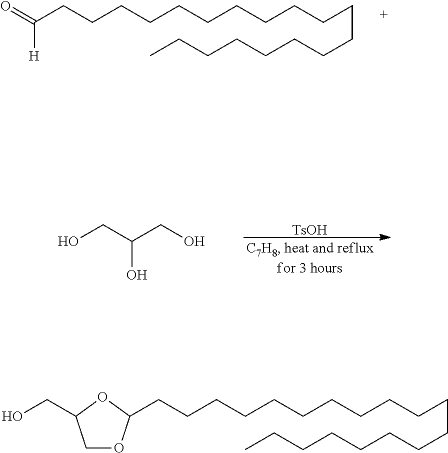 [SMILES: CCCCCCCCCCCCCCCCCCCC=O ; OCC(O)CO ; "TsOH" ; CCCCCCCCCCCCCCCCCCCC1OCC(CO)O1]

In order to purify the compound of formula (I), the mixture was dissolved at 5 ml of hexane and then separated the white compound of formula (I) about total 260 mg by silica gel column chromatography, wherein the elution buffer used in the silica gel column chromatography was hexane/ethyl acetate (9:1).

### Example 2

### Purification of the Active Component for Liver Protection from Clams

With reference to the description of the related prior art, removing the clamshells from 65 kg of clams to have clam bodies were about 27.7 kg. After drying by heated-wind, the weight of the dried clam bodies was about 2.5 kg, and then extracted by soaking into alcohol which weight was threefold as much as the weight of the clam dried bodies. Obtaining a clam-alcohol extract was about 1 kg.

900 grams of the clam-alcohol extract, water and ethyl acetate were mixed at the volume ratio 1:1:1, and then separating and extracting it by separating funnel to obtain an ethyl acetate layer. The ethyl acetate layer was condensed and frozen to obtain a clam-alcohol-ethyl-acetate extract. 900 grams of the ethyl acetate layer was separated by using chromatography column with the porous silicone gel (40˜63 pores), and sequentially eluted with the solutions of hexanol and ethyl acetate at the volume ratio of 10:1, 5:1, 3:1, 1:1, 1:3, 1:5 and 1:10 and methanol, and then 18 fragments were taken. The 18 fragments were analyzed by thin layer chromatography (TLC) to isolate an active component about 1 gram, wherein, liver protection effect of the active component was disclosed in Taiwan patent no. 1340747, so there will be no more description about this matter.

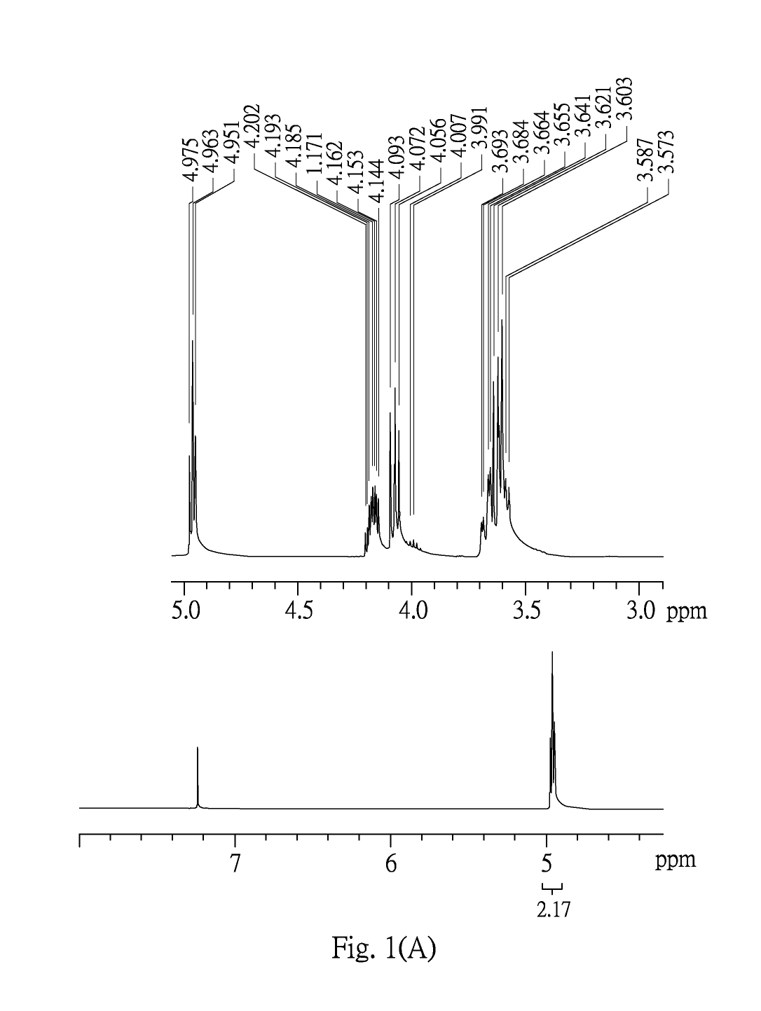

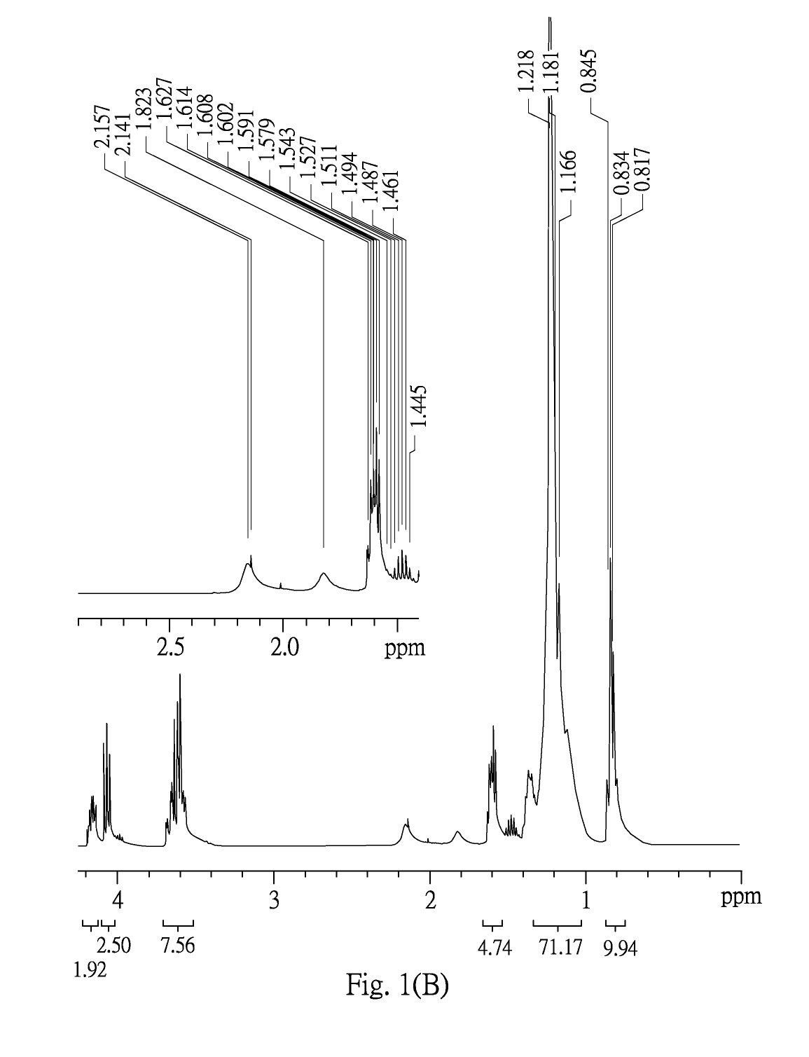

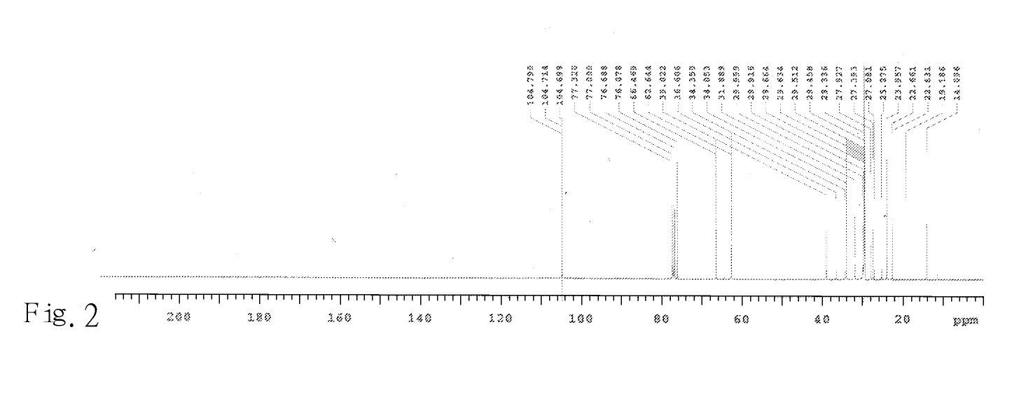

The active component has the following characteristics: white and solid, the melting point about 51° C., the specific rotatory power: [α]D25+1.15 (c=0.75, methanol). Rf value of the active component is 0.3 by TLC under the condition of hexanol and ethyl acetate at the volume ratio of 4:1. Moreover, analyzed by hydrogen nuclear magnetic resonance spectroscopy (¹H-NMR) and carbon-13 nuclear magnetic resonance spectroscopy (¹³C-NMR), the spectrums of the active component are shown as FIG. 1 and FIG. 2, wherein deuterated chloroform (CDCl₃) was used as a solvent and the frequency was 400 MHz.

According to FIG. 1, it shows that δ_{H} values of the active component were 4.96 (1H, t, J=4.8 Hz, H-2), 4.17 (1H, m, H-4), 4.08 (1H, dd, J=8.4, 6.4 Hz, H-5a), 3.67 (1H, dd, J=11.6, 3.6 Hz, H-6b), 3.62 (1H, dd, J=8.4, 7.2 Hz, H-5b), 3.58 (1H, dd, J=11.6, 5.6 Hz, H-6b), 2.15 (1H, br. s, 6-OH), 1.59-1.63 (2H, m, H-1′), 1.27-1.43 (2H, m, H-2′), 1.01-1.25 (32H, br s, H-3′-18′), 0.87 (t, 3H, J=6.6 Hz, H-19′). According to FIG. 2, it shows that δ_{c} values of the active component were 104.7 (C-2), 76.1 (C-5), 66.5 (C-4), 62.6 (C-6), 34.1 (C-1′), 31.9 (C-17′), 29.3-29.9 (C-3′-16′), 24.0 (C-2′), 22.7 (C-18′), 14.1 (C-19′).

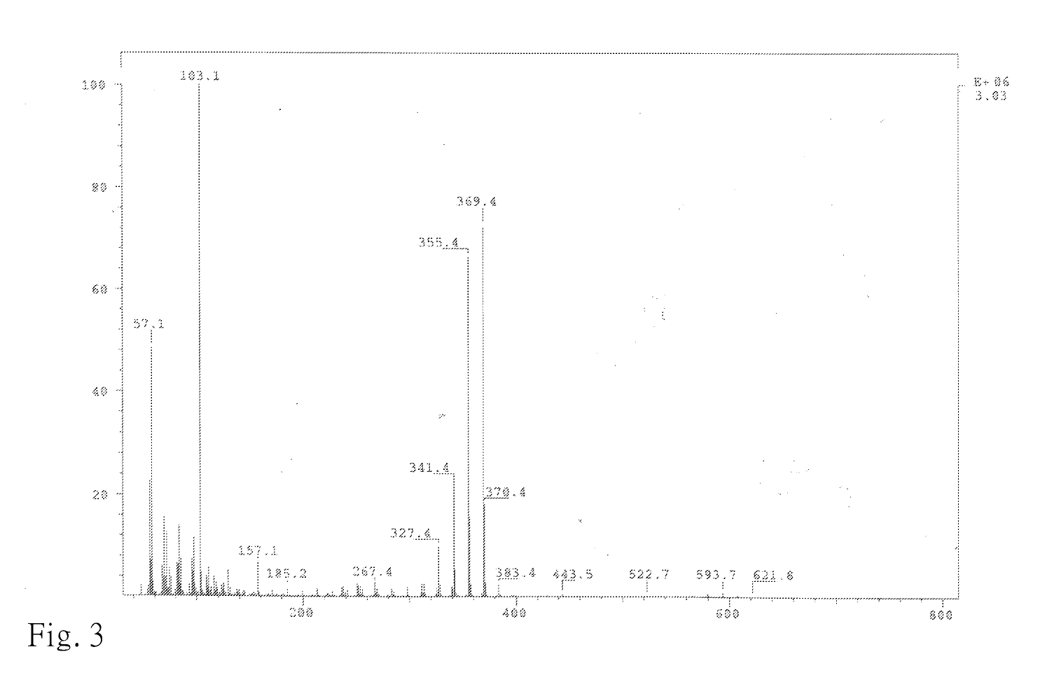

Furthermore, the active component was analyzed by electron impact-mass spectrophotometer (EI-MS), and the result is shown as FIG. 3. FIG. 3 disclosed that molecular ion peak [M]+charge-to-mass ratio and relative intensity of the active component, and it shows EI-MS: m/z (%): 370(20) [M]⁺, 355(65), 341(22), 327(11), 267(5), 185(3), 157(9), 103(100), 57(51).

Based on the results of example 1 and 2, it can figure out that structure of compound of formula (I) from the method of this present invention is the same as the active component produced from example 2 by comparing the compound of formula (I) from example 1 and the active component from example 2. In other words, compound of formula (I) from this present invention has function for preventing or treating liver diseases, especially the liver fibrosis related liver diseases. Therefore, effective amount of compound of formula (I) can be taken as a functional material of pharmaceutical composition or daily nutrition supplement.

Furthermore, according to the method revealed in example 2, using 27.7 kg of clam bodies as material only can produce 1 gram of the active component. The productivity of the method in example 2 was merely 0.004%. Comparison of the method in example 2, the method of this present invention only using 2 kg material and it can obtain 260 mg of compound of formula (I). The productivity of the method from this present invention was 13% and it was obviously greater than the conventional method. Accordingly, the method of this present invention can effectively increase productivity of compound of formula (I), at the same time reduce costs of production.

It should be understood that the above-mentioned detailed description and specific examples, while indicating preferred embodiments of the invention, are given by way of illustration only. Various changes and modifications within the spirit and scope of the invention will become apparent to those skilled in the art from this detailed description. All such modifications as would be obvious to one skilled in the art are intended to be included within the scope of the following claims.

What is claimed is:

1. A compound of formula (I) 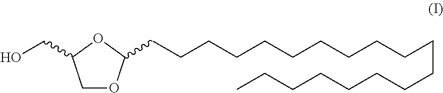 [SMILES: CCCCCCCCCCCCCCCCCCCC1OCC(CO)O1]
or pharmaceutically acceptable salts thereof.

2. A method for producing a compound of formula (1) or pharmaceutically acceptable salts thereof according to claim 1, comprising obtaining a mixture containing the compound of formula (I) is produced by acetalization reaction of the compounds of formula (II): 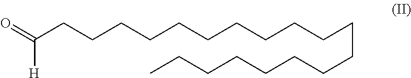 [SMILES: CCCCCCCCCCCCCCCCCCCC=O]
and formula (III):

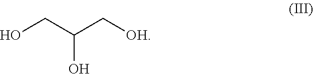

3. The method according to claim 2, wherein the compound of formula (II) is produced by oxidation reaction of the compound of formula (IV): 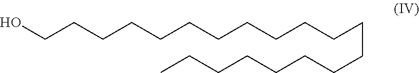 [SMILES: CCCCCCCCCCCCCCCCCCCCO]
and the pyridinium chlorochromate in organic solvent.

4. The method according to claim 2, wherein an acidic catalyst is used during the acetalization reaction.

5. The method according to claim 2, wherein the mixture containing the compound of formula (I) can be purified by separation process.

6. A method for treating liver disease, comprising administering a subject an effective amount of a compound of formula (I) or pharmaceutically acceptable salts thereof according to claim 1.

7. The method according to claim 6, wherein the liver disease is liver fibrosis related disease.
